(19) United States
(12) Patent Application Publication
(10) Pub. No.: US 2010/0160862 A1
(43) Pub. Date: Jun. 24, 2010

(54) VARIABLE STIFFNESS INTRODUCER SHEATH WITH TRANSITION ZONE

(75) Inventors: William L. Howat, Laconia, NH (US); Thomas A. Osborne, Bloomington, IN (US)

Correspondence Address: BRINKS HOFER GILSON & LIONE/INDY/COOK;BRINKS HOFER GILSON & LIONE, CAPITAL CENTER, SUITE 1100, 201 NORTH ILLINOIS STREET, INDIANAPOLIS, IN, 46204-4220, US

(73) Assignee: Cook Incorporated, Bloomington, IN (US)

(21) Appl. No.: 12/341,459

(22) Filed: Dec. 22, 2008

(51) Int. Cl. A61M 5/00; B32B 37/06

(52) U.S. Cl. 604/164.01; 156/86

(57) ABSTRACT

An introducer sheath includes a lubricious inner liner having a passageway extending longitudinally therethrough, a reinforcing member positioned over the inner liner, and an outer jacket positioned longitudinally over the reinforcing member and the inner liner. The outer jacket has a higher durometer proximal portion, a lower durometer distal portion, and a transition zone between the proximal portion and the distal portion. The transition zone has a variable durometer from a junction with the outer jacket proximal portion to a junction with the outer jacket distal portion, to provide a transition between the higher durometer proximal portion and the lower durometer distal portion.

### BACKGROUND

1. Technical Field

This invention relates to a tubular medical device suitable for accessing a target site within the body of a patient, and more particularly, to an introducer sheath having segments of different stiffness separated by a transition zone.

2. Background Information

Introducer sheaths are in widespread use in the medical field for delivering a medical interventional device, such as a stent, to a target site within a bodily passageway of a patient, such as the vasculature. In order to reach the target site, the sheaths are often required to traverse tortuous pathways having sharp bends and angles. In some instances, and particularly when traversing such tortuous pathways, the sheaths exhibit a tendency to kink. Kinking reduces, and at times collapses, the effective inner diameter of the sheath. When this occurs, the sheath becomes unsuitable for its intended use.

The tendency of a sheath to kink is increased when the sheath is used to introduce the interventional device into one of the many smaller vessels that branch off from major vessels. In this event, the sheath may have insufficient flexibility at the very point where flexibility is most desired in order to enable proper positioning of the interventional device. In order to traverse the narrow confines of, e.g., the vascular system, the introducer sheath is typically formed of thin-wall construction. However, thin wall sheaths often have difficulty tracking narrow vessels, and exhibit an increased propensity to kink. Increasing the thickness of the sheath wall can minimally improve the level of kink resistance, as well as the trackability of the sheath. Any such increase in thickness, however, is inherently undesirable. The thickness increase limits the ability of the sheath to enter a narrow vessel, while at the same time reducing the diameter of the lumen when compared to the lumen of an otherwise similar thin-walled sheath. In addition, a larger diameter sheath necessitates the use of a larger entry opening than would otherwise be required or desirable.

One introducer sheath with improved kink resistance is disclosed in U.S. Pat. No. 5,380,304 to Parker, incorporated by reference herein. The introducer sheath described in the '304 patent comprises an inner liner formed of a lubricious fluoropolymer, such as polytetrafluoroethylene (PTFE). A coil is fitted around the inner PTFE liner, and an outer jacket comprising a heat-formable material, such as nylon or a polyether block amide, surrounds the inner liner and coil. The heat-formable material is heat shrunk onto the PTFE outer surface by enveloping it in a heat shrink enclosure, and heating the entire assembly until the material melts. As the heat-formable material melts, it flows between the spacings of the coil turns, and bonds to the outer diameter of the PTFE layer. The use of the coil in this device reinforces the sheath wall, and provides enhanced kink-resistance to an otherwise thin-walled introducer sheath.

The introducer sheath described in the '304 patent has proven to be particularly effective in allowing the medical professional to deliver medical devices and medicaments to remote areas of a patient's vasculature without kinking. In order to minimize the cross-sectional profile (i.e., the outer diameter) of the sheath, the coil is generally formed of flat wire, as shown in FIG. 2 of the patent. By utilizing a flat wire coil, the sheath achieves a high level of kink resistance, and at the same time, maintains a low cross-sectional profile. The sheath described in the '304 patent enables the physician to routinely access, without kinking, target areas of the vasculature that had previously been difficult, or impossible, to reach.

With the continuous advances in the medical arts, additional features have been developed to enhance the use of such introducer sheaths. For example, introducer sheaths have been developed wherein a proximal portion of the sheath having a relatively high stiffness is bonded to a distal portion having a relatively low stiffness. One such sheath is disclosed in U.S. Patent Publication No. 2001/0034514, incorporated by reference herein. Since the distal portion of the sheath has a lower stiffness (and therefore is more flexible) than the proximal portion, the sheath is able to traverse portions of the anatomy that would have been difficult, if not impossible, to traverse with stiffer sheaths. Since the proximal portion has a higher stiffness (and is therefore less flexible) than the distal portion, the sheath maintains the trackability to traverse tortuous areas of the anatomy. This presence of the coil reinforcement also enables this sheath to be kink resistant through a wide range of bending angles.

Although the sheath described in the patent publication referenced above exhibits improved trackability in many instances when compared to a sheath not having portions of variable stiffness, the presence of the adjacent sheath portions of variable stiffness has a potential to cause difficulty when attempting to introduce the sheath into a tortuous portion of the vasculature. If the respective sheath portions result in an abrupt change in stiffness at the area of joinder of the (high stiffness) proximal portion and the (low stiffness) distal portion, the sheath portion may “elbow” at the area of joinder when subjected to a bending force. As a result, a stress riser may be generated at the area of bending. A stress riser comprises a weakened or high stress area of the sheath which may cause the sheath to undesirably bend or kink during passage through the vessel.

It is desired to provide an introducer sheath that overcomes the problems encountered with prior art sheaths that have discrete sheath portions of different stiffnesses, and that minimizes the possibility of a stress riser being formed between the sheath portions.

### BRIEF DESCRIPTION OF THE DRAWINGS

FIG. 1 is a side view of a flexible, kink-resistant introducer sheath of a type known in the prior art, shown in combination with a dilator and a hub;

FIG. 2 is a longitudinal cross-sectional view of a segment of the wall of the prior art introducer sheath of FIG. 1, taken along line 2-2 of FIG. 1;

FIG. 2A is a longitudinal cross-sectional view as in FIG. 2, illustrating the bending of the sheath upon exposure to a bending force;

FIG. 3 is a longitudinal sectional view of a portion of an introducer sheath according to an embodiment of the present invention; and

FIG. 4 is an outer, side view of a portion of an introducer sheath according to an embodiment of the present invention, illustrating a bending of the sheath as it passes through a curved body passageway.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In the following discussion, the terms “proximal” and “distal” will be used to describe the opposing axial ends of the inventive sheath, as well as the axial ends of various component features. The term “proximal” is used in its conventional sense to refer to the end of the sheath (or component thereof) that is closest to the operator during use of the apparatus. The term “distal” is used in its conventional sense to refer to the end of the sheath (or component thereof) that is initially inserted into the patient, or that is closest to the patient during use.

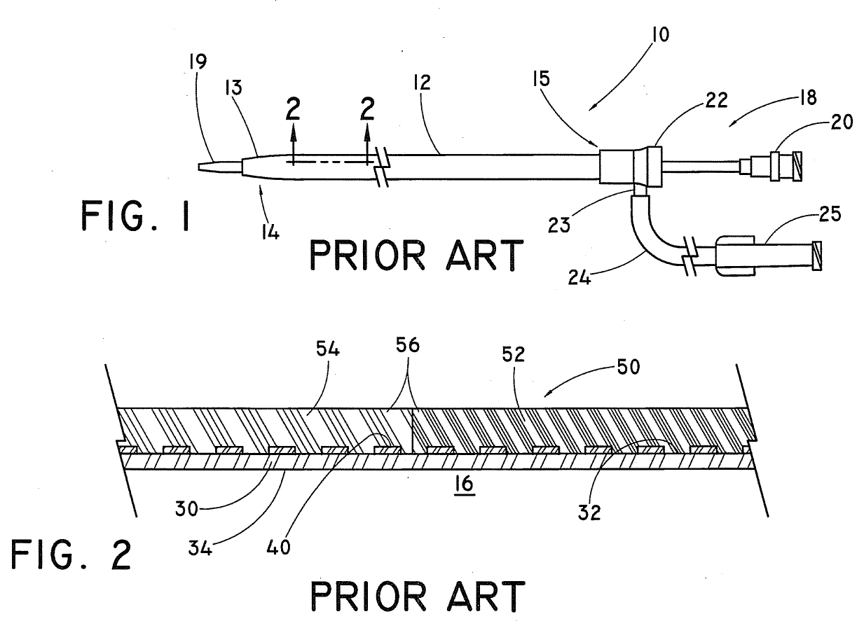

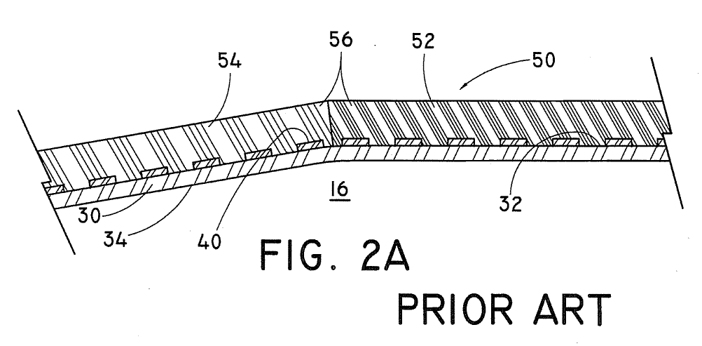

FIG. 1 shows an illustrative flexible introducer sheath 10 of a type known in the art. Introducer sheath 10 includes an outer tube 12, having a distal portion 13 and a proximal portion 15. Preferably, distal portion 13 tapers to a tapered distal end 14. An inner passageway 16 (FIG. 2) extends longitudinally through sheath 10 in well-known fashion.

In FIG. 1, sheath 10 is shown in combination with an optional dilator 18 and connector hub 22. Dilators and connector hubs for use with introducer devices, such as sheath 10, are well known, and the particular dilator and hub illustrated in FIG. 1 may be replaced with various other dilators and hubs known in the art. As shown herein, dilator 18 extends longitudinally through the passageway of the sheath. The dilator includes a tapered distal end 19 for accessing and dilating a vascular access site, e.g., over a wire guide (not shown) by any conventional vascular access technique, such as the well-known Seldinger technique. A Luer lock connector 20 may be attached at the proximal end of the dilator for connection to a syringe or other medical apparatus in well known fashion.

Connector hub 22 is attached about the proximal end of the sheath during use. Connector hub 22 may include one or more conventional silicone disks (not shown) for preventing the backflow of fluids therethrough. Connector hub 22 may also include a side arm 23, to which a polymeric tube 24 and a conventional connector 25 may be connected for introducing and aspirating fluids therethrough in conventional fashion.

FIG. 2 is a longitudinal cross-sectional view of a segment of the wall of the prior art introducer sheath 10 of FIG. 1. As illustrated, sheath 10 comprises an inner liner 30, having a radially outer surface 32 and a radially inner surface 34. A conventional reinforcing member, such as coil 40, is wound or otherwise fitted around the radially outer surface 32 of liner 30. A polymeric outer layer or jacket 50 is bonded to the outer surface 32 of inner liner 30 through the spaced turns of the coil 40.

Liner 30 is typically formed of a lubricious fluoropolymer, such as polytetrafluoroethylene (PTFE) or fluorinated ethylene propylene (FEP). The lubricious material provides a slippery, low friction inner surface 34 to ease insertion and/or withdrawal through passageway 16 of the dilator or of a medical interventional device, such as a stent. Liner 30 generally has a substantially uniform inner diameter that extends the entire length of passageway 16, to allow passage therethrough of an interventional device having the largest possible diameter. The radially outer surface 32 of liner 30 is typically roughened in any conventional manner, such as by chemical etching, to form an irregular surface to facilitate bonding with outer polymeric jacket 50. The wall of the inner liner will also generally have sufficient structural integrity to prevent the coil turns from protruding into inner passageway 16.

Coil 40 is typically formed from well-known materials commonly utilized for such purposes in the medical arts, such as a metal, a metal alloy (e.g., stainless steel or a shape memory composition such as nitinol), a multi-filar material, or a composite material. In order to minimize the cross-sectional profile (i.e., outer diameter) of the sheath, the coil is generally formed from a conventional flat wire construction.

Outer jacket 50 is typically formed from a polymeric material capable of forming a thermal bond with the lubricious inner liner material. Examples of such materials commonly utilized in the art include a polyether block amide, a polyamide (e.g., nylon), and polyurethane. In the prior art sheath shown above, outer jacket 50 comprises a relatively stiff proximal portion 52 and a relatively flexible distal portion 54. This is typically accomplished by providing a polymeric material of relatively high stiffness, or durometer, at the proximal end, and a polymeric material of a relatively low stiffness, or durometer, at the distal end.

One common method of forming the prior art sheath 10 will now be described. Initially, the liner 30 is positioned over a supporting mandrel. The reinforcing member, such as coil 40, is then wrapped, wound, compression fitted, or otherwise positioned around the outer surface 32 of inner liner 30. Outer jacket proximal portion 52 and distal portion 54 are then sequentially slid over the coil and inner liner such that their adjacent ends are in general abutment with each other. The entire assembly (the inner liner, coil, and outer jacket portions) is then placed in a heat shrink enclosure, and the heat shrink enclosure is heated in an oven to at least partially melt the outer jacket composition. The melted composition flows between the turns of the coil, and bonds to the roughened outer surface of the inner liner. The melted outer jacket composition also causes the abutted axial ends of respective outer proximal and distal outer jacket portions 52, 54 to bond to each other. Following formation of the bonds as described above, the assembly is allowed to cool, and thereafter removed from the heat shrink enclosure. The mandrel is then removed from the inner liner. Further details of the prior art processes may be found in the incorporated-by-reference patent documents.

By bonding together the adjacent ends of higher durometer outer jacket portion 52 and lower durometer outer jacket portion 54 as described, a stress riser may be formed at the area of joinder 56 due to the abrupt change in stiffness that may be present. When present, a stress riser comprises a weakened or high stress area of the sheath. The weakened or high stress area may cause the sheath to undesirably bend during passage through the vessel, and can result in the formation of a kink in the sheath. Ultimately, the weakened area can lead to premature failure of the bond.

One example of the type of bending that may occur in such instances is illustrated in FIG. 2A. In this example, lower durometer portion 54 has undergone bending with reference to higher durometer portion 52 upon exposure to a bending force. Those skilled in the art will appreciate that the particular bending angle shown in FIG. 2A is merely one example of many possible angles (larger or smaller) that result from such exposure. The actual bending angle in any particular instance will depend on factors such as the respective durometers of portions 52, 54, and the geometry of the particular body passageway through which the sheath is passed, among others.

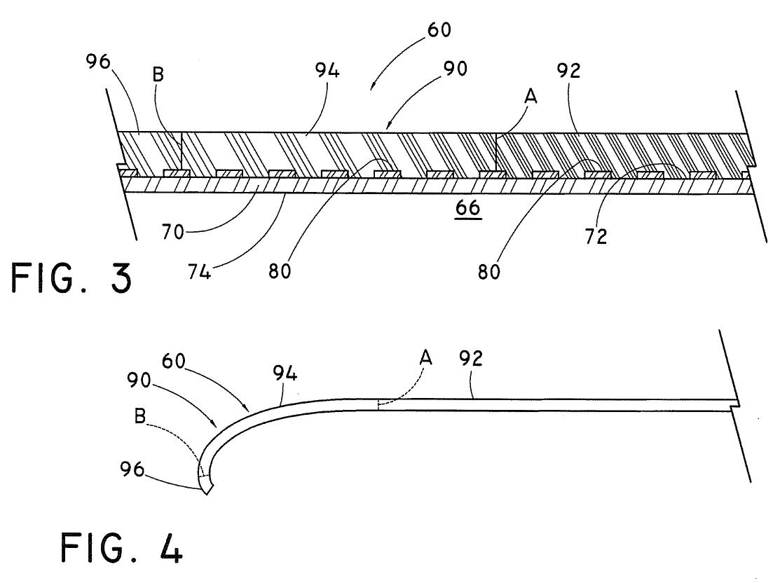

The variable stiffness introducer sheath disclosed herein is similar in some respects to the variable stiffness prior art sheath described above. A longitudinal sectional view of a length of an introducer sheath 60 according to an embodiment of the present invention is shown in FIG. 3. FIG. 4 illustrates an outer, side view of a portion of an introducer sheath according to an embodiment of the present invention, illustrating the bending of the sheath as it passes through a curved body passageway. As noted in FIG. 4, the inventive sheath does not include a sharp bend, or elbow, of the type exhibited with prior art structures, e.g., FIG. 2A, as it is curved for passage through a targeted pathway in the body of a patient.

The length of sheath 60 visible in FIG. 3 corresponds generally to the position of the length of the prior art sheath in the vicinity of the area of joinder 56, as illustrated in FIG. 2. The outer tubular portion of sheath 60 is similar to the visible portion of prior art sheath 10 at line 2-2, as shown in FIG. 1. In use, sheath 60 may also be provided with an optional dilator and connector hub, as shown in the prior art embodiment of FIG. 1.

In the embodiment shown, sheath 60 comprises an inner liner 70, having a radially outer surface 72 and a radially inner surface 74. A conventional reinforcing member, such as coil 80, may be wound, compression fitted, or otherwise positioned around the radially outer surface 72 of liner 70, in the same manner as coil 40 of the prior art structure. A polymeric outer jacket 90 may be bonded to the outer surface 72 of inner liner 70 through the spaced turns of the coil 80, in the same manner as in the prior art structure referenced above. Outer jacket 90 will be further described herein.

Liner 70 is typically formed of a lubricious fluoropolymer, such as polytetrafluoroethylene (PTFE) or fluorinated ethylene propylene (FEP), in the nature of the prior art structure. Preferably, liner 70 has a substantially uniform inner diameter that extends the entire length of an inner passageway 66 of the liner. The radially outer surface 72 of liner 70 is preferably etched or otherwise roughened in conventional fashion to form an irregular surface for facilitating bonding with the outer polymeric jacket 90. The wall of the inner liner will preferably have sufficient structural integrity to prevent the coil turns from protruding into inner passageway 66. Coil 80 may be formed of any compositions commonly utilized for such purposes in the medical arts, including those compositions recited above with reference to the prior art sheaths.

Outer jacket 90 is typically formed from extrudable polymeric materials of a type commonly used in the art with introducer sheaths. Non-limiting examples of such outer jacket materials include a polyether block amide, a polyamide (e.g., nylon), and polyurethane. Outer jacket 90 has discrete portions, or segments, of specified durometer. In the embodiment shown, outer jacket 90 comprises a proximal portion 92, a transition portion or zone 94, and a distal portion 96. Proximal portion 92 has a relatively high durometer of, for example, about 60-80 on the Shore D scale. Distal portion 96 has a relatively low durometer, of, for example, about 25-50 on the Shore D scale. The durometers recited hereinabove are provided for purposes of illustration only, and not by way of limitation.

Transition zone 94 has a variable durometer, ranging from a relatively high durometer at a point of joinder A with the proximal portion 92 to a relatively low durometer at a point of joinder B with the distal portion 96. Preferably, the relatively high durometer at point A referenced above is the same as, or slightly lower than, the durometer of the proximal portion. Thus, for example, when the durometer of proximal portion 92 is 70, the durometer of transition zone 94 at point of joinder A would preferably be about 70, or slightly lower than 70.

Similarly, the relatively low durometer at point B referenced above would be the same as, or slightly higher than, the durometer of the distal portion 96. Thus, for example, when the durometer of distal portion 96 is 40, the relatively low durometer at point B would preferably be 40, or slightly higher than 40.

In a preferred embodiment, the durometer of transition zone 94 will exhibit a gradual and continuous decrease at a generally constant rate along the length of the transition zone from the high durometer point A (e.g., durometer of about 70) to low durometer point B (e.g., durometer of about 40). This gradual decrease of durometer minimizes, or eliminates altogether, the possibility of formation of a stress riser of the type that may be formed in many prior art structures at a junction between sheath portions of different durometers. Although a gradual and continuous decrease in durometer of transition zone 94 between points A and B is generally preferred, such a gradual and continuous durometer decrease may not be necessary in all instances. In such case, a designated length of transition zone 94 may exhibit a greater rate of decrease of durometer than another length of zone 94.

Those skilled in the art will appreciate that an introducer sheath may be formed to have any length suitable for its intended use. Typically, such sheaths are between about 15 and 150 cm in length. In most cases, the proximal portion of the sheath comprises the major length of the sheath. Thus, for example, with a sheath having a length of about 40 cm, the proximal portion may have a length of at least about 25-30 cm. In this case, the transition zone may have a length of about 5-10 cm. The distal portion may have a length of about 1 cm or less, such as about 2-3 mm. Those skilled in the art can readily optimize the various lengths depending upon the intended use of the sheath, the durometer difference between the proximal and distal portions, and other known design features that may be beneficial for a particular purpose.

Although the embodiment of the inventive sheath 60 illustrated in FIGS. 3 and 4 includes an outer jacket 90 having a proximal portion 92, a distal portion 96, and a transition zone 94 positioned therebetween, the invention is not limited to this arrangement. Rather, the outer jacket can include any number of portions, or segments, of discrete durometer, with a separate transition zone between any two segments of different durometer.

The following discussion describes a preferred way of forming a sheath according to an embodiment of the present invention. Initially, an outer jacket 90 is formed. The outer jacket preferably has respective high and low durometer portions, and a transition zone therebetween. A particularly preferred way of forming the outer jacket is by a continuous intermittent extrusion process. Continuous intermittent extrusion processes enable the continuous extrusion, without bonding, of a tubular structure having portions of different durometer. With continuous intermittent extrusion, the tubular outer jacket can be extruded to be, e.g., rigid or semi-rigid at one end and flexible at the other end. Preferably, the jacket is extruded such that it has a gradual durometer decrease over a defined length of the sheath, such as along transition zone 94 as described in the discussion concerning FIGS. 3 and 4. Providing a gradual durometer decrease along the length of the transition zone generally eliminates the possibility of a stress riser being formed along the length of the transition zone.

Thus, for example, a tubular material can be extruded to have a proximal portion of a desired length having a discrete high durometer, a distal portion of a desired length having a discrete low durometer, and a transition zone of a desired length therebetween. Preferably, the durometer of the transition zone will decrease along the length of the zone from the (higher) durometer of the proximal portion to the (lower) durometer of the distal portion. As a further alternative, the transition portion can extend all the way to the distal end of the sheath. In this event, a separate distal tip portion of a discrete durometer need not be provided.

Extrusion techniques capable of such continuous variable extrusion are now well known in the art, and a skilled artisan can readily fabricate an extrusion having virtually any number of segments having dimensions (both length and durometer) as may be desired for a particular application. With continuous extrusion, the sheath can be formed to eliminate areas of high stress that are common in many conventional devices at the bonding site where high and low durometer segments are joined together.

Following the continuous intermittent extrusion process as described, the respective axial ends of the resulting tubular material may be cut to length such that the proximal portion and the distal portion each have a defined length. Extruded tubular sheaths having segments of varying lengths and durometer are now commercially available, and may be made to specification. Among other sources, such sheaths may be obtained from Putnam Plastics, Co., of Dayville, Conn.

Following fabrication of the outer jacket, the sheath may then be formed in the following manner. The inner sheath liner 70 is initially positioned over a supporting mandrel. The reinforcing member, such as coil 80, is then wrapped, wound, compression fitted, or otherwise positioned around the outer surface of the inner liner. The outer jacket 90 having the variable stiffness portions as described is then positioned over the coil and inner liner, and an assembly comprising the inner liner, coil, and outer jacket is placed in a suitable heat shrink enclosure formed of a material, such as FEP, that is capable of being heated to a temperature in excess of the melt temperature of the outer jacket composition.

The heat shrink enclosure with the assembly therein is placed in an oven, and heated to a temperature sufficient to melt the outer jacket composition. As the heat shrink enclosure shrinks upon the application of heat, the melted outer jacket is compressed by the shrinking FEP enclosure between the turns of the coil to bond with the roughened outer surface of the inner liner in well-known fashion. The assembly is allowed to cool, and thereafter removed from the heat shrink enclosure. The supporting mandrel is slid out from the interior of the sheath. The respective longitudinal ends of the sheath may be trimmed to a desired length.

The heat shrink operation described above provides an effective manner of forming the bond between the outer jacket and the inner liner. However, those skilled in the art will appreciate that the heat shrink is not required in all instances. Thus, for example, in one alternative method for forming the sheath, the outer jacket need not be separately extruded for later application over an inner liner and coil in the manner previously described. Rather, in this alternative, the outer jacket can be directly extruded onto a wire (mandrel), upon which the inner liner and coil have already been positioned. In this alternative, the extruded outer jacket retains a melt temperature upon its deposition over the inner liner sufficient to form a bond therebetween. Those skilled in the art will appreciate that the methods described above are examples only of suitable methods for forming the sheath, and that other methods may be substituted, all of which are considered within the scope of the invention.

Although the sheath described hereinabove preferably utilizes a conventional flat wire coil reinforcement, the teachings of the present invention are also applicable to sheaths having coils of other cross-sectional geometries. In addition, the teachings are also applicable to sheaths having other reinforcing structures disposed therewithin, such as a braided reinforcement formed of interwoven wires.

Those skilled in the art will appreciate that all dimensions, durometers, compositions, etc., described herein are exemplary only, and that other appropriate dimensions, durometers, compositions, etc., may be substituted in an appropriate case. Additionally, other features commonly utilized in medical sheaths, such as radiopaque markers, rings, coatings, etc., may also be incorporated into the inventive structure in well-known manner.

It is therefore intended that the foregoing detailed description be regarded as illustrative rather than limiting, and that it be understood that it is the following claims, including all equivalents, that are intended to define the spirit and scope of this invention.

1. An introducer sheath comprising:
a lubricious inner liner having a passageway extending longitudinally therethrough, said inner liner having an outer surface;
a reinforcing member positioned over said inner liner; and
an outer jacket positioned longitudinally over said reinforcing member and said inner liner, said outer jacket having a higher durometer proximal portion, a lower durometer distal portion, and a transition zone between said proximal portion and said distal portion, said transition zone having a proximal end and a distal end, said transition zone varying in durometer from said transition zone proximal end to distal end to provide a transition between said higher durometer proximal portion and said lower durometer distal portion.

2. The introducer sheath of claim 1, wherein the transition zone durometer at said proximal end is substantially the same as the durometer of said outer jacket proximal portion.

3. The introducer sheath of claim 1, wherein the transition zone durometer at said distal end is substantially the same as the durometer of said outer jacket distal portion.

4. The introducer sheath of claim 1, wherein the transition zone durometer at said proximal end is substantially the same as the durometer of said outer jacket proximal portion, and the transition zone durometer at said distal end is substantially the same as the durometer of said outer jacket distal portion.

5. The introducer sheath of claim 4, wherein said transition zone exhibits a gradual decrease in durometer from said proximal end of said transition zone to said distal end.

6. The introducer sheath of claim 1, wherein said transition zone varies in durometer by at least about 30 on the Shore D scale from said transition zone proximal end to distal end.

7. The introducer sheath of claim 1, wherein said reinforcing member comprises a flat wire coil.

8. The introducer sheath of claim 7, wherein said outer jacket comprises a polyether block amide, a polyamide or a polyurethane.

9. An introducer sheath comprising:
a lubricious inner liner having a passageway extending longitudinally therethrough, said inner liner having an outer surface;
a reinforcing member positioned over said inner liner outer surface; and
an outer jacket positioned longitudinally over said reinforcing member and said inner liner, said outer jacket having a proximal portion, and having a transition zone extending in a distal direction therefrom, said proximal portion having a first durometer, said transition zone having a length and having a variable durometer along said length, said variable durometer decreasing along said length from a durometer substantially similar to the first durometer at a junction between said proximal portion and said transition zone.

10. The introducer sheath of claim 9, wherein said transition zone durometer gradually and continuously decreases along said length.

11. The introducer sheath of claim 9, wherein said outer jacket further comprises a distal tip portion, said distal tip portion having a second durometer, and wherein said variable durometer decreases along said transition zone length from said durometer substantially similar to the first durometer at said junction with said proximal portion to a durometer substantially similar to said second durometer at a junction with said distal tip portion.

12. The introducer sheath of claim 9, wherein said distal tip portion does not exceed 1 cm in length.

13. The introducer sheath of claim 12, wherein said durometer of said transition zone gradually decreases along said transition zone length.

14. The introducer sheath of claim 9, wherein said inner liner comprises PTFE, said reinforcing member comprises a flat wire coil, and said outer jacket comprises at least one of a polyether block amide, a polyamide, and polyurethane.

15. A method of forming an introducer sheath, comprising:
providing a generally tubular inner liner;
positioning a reinforcing member over a length of the inner liner;
positioning an outer jacket over the reinforcing member and the inner liner, the outer jacket having a higher durometer proximal portion, a lower durometer distal portion, and a transition zone between said proximal portion and said distal portion, said transition zone having a proximal end and a distal end, said transition zone varying in durometer from said transition zone proximal end to distal end to provide a transition between said higher durometer proximal portion and said lower durometer distal portion; and
bonding said outer jacket to an outer surface of said inner liner.

16. The method of claim 15, wherein the transition zone durometer at said proximal end is substantially the same as the durometer of said outer jacket proximal portion, and the transition zone durometer at said distal end is substantially the same as the durometer of said outer jacket distal portion.

17. The method of claim 16, wherein said transition zone exhibits a gradual decrease in durometer from said proximal end of said transition zone to said distal end.

18. The method of claim 15, wherein the outer jacket is formed by continuous intermittent extrusion.

19. The method of claim 15, wherein said inner liner comprises PTFE, and said reinforcing member comprises a coil having a plurality of coil turns.

20. The method of claim 19, wherein inner liner having said coil and outer jacket positioned thereover is placed in a heat shrink enclosure, and said enclosure is exposed to a sufficient amount of heat to thermally bond the outer jacket to said inner liner outer surface between said coil turns.
